(19) United States
(12) Patent Application Publication
(10) Pub. No.: US 2011/0015245 A1
(43) Pub. Date: Jan. 20, 2011

(54) BENDAMUSTINE AMPHIPHILIC CATIONIC COMPOSITIONS

(76) Inventors: Valery Alakhov, Ile Bizard (CA); Grzegroz Pietrzynski, Montreal (CA); Patel Kishore, Pierrefonds (CA); Thomasz Popek, Pointe-Claire (CA)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 12/838,940

(22) Filed: Jul. 19, 2010

Related U.S. Application Data
(60) Provisional application No. 61/271,366, filed on Jul. 20, 2009.

(51) Int. Cl. A61K 31/4184; A61P 35/00; A61P 35/04

(52) U.S. Cl. 514/394

(57) ABSTRACT

The present invention is directed to pharmaceutical compositions comprising bendamustine and one or more amphiphilic cationic compounds, which self assemble to form aggregates. The exhibiting enhanced stability in aqueous solutions, including plasma. The unexpectedly enhanced stability afforded by such aggregates permits patients to be treated with bendamustine in lower and/or with less frequent dosages or to improve its therapeutic effect while using the same as presently used treatment protocol.

### CROSS REFERENCE TO RELATED APPLICATION

This application claims the benefit of U.S. Provisional Patent Application No. 61/271,366, filed Jul. 20, 2009, the entirety of which is hereby incorporated by reference into this application.

### FIELD OF THE INVENTION

The present invention is directed to pharmaceutical compositions comprising bendamustine and amphiphilic cationic compounds, in particular to an aggregate form of the composition comprising bendamustine and one or more amphiphilic cationic compounds, which aggregates exhibit enhanced stability in aqueous solutions, including plasma.

### BACKGROUND OF THE INVENTION

Bendamustine, 4-[5-[BiS(2-chloroethyl)amino]-1-methylbenzimidazol-2-yl]butanoic acid, is used in the treatment of leukemia and certain lymphomas. However, this compound has limited chemical stability in plasma, thereby requiring high or repeated doses in order to achieve a therapeutic effect. United States Patent Application Publication No. 2006/0159713 (Brittain et al.) indicates that, once reconstituted into aqueous solutions, bendamustine quickly degrades and must therefore be administered to patients as quickly as possible. Maas et al.; “Stabililitat von Bendamustinhydrochlorid in Infusionslosungen”; Pharmazie 49:775-7 (1994) discloses that bendamustine hydrochloride is stable for only 9 hours at 23° C. in saline solution.

Attempts have been made to increase the stability of bendamustine by complexing such molecule with polymeric materials. However, the approaches taken have only achieved marginal success. Pencheva et al.; “HPLC study on the stability of bendamustine hydrochloride immobilized onto polyphosphoesters; J. Pharma. Biomed. Anal; (2008), attempted to improve the stability of bendamustine by complexing such compound with polyphosphoesters. However, FIG. 2 of such article shows that even the most stable complex decreases by a full log point (90%) in about 45 minutes at pH 7.

Evjen; “Development of Improved Bendamustin-Liposomes”; Masters Thesis; University of Tromso (2007), employed dual asymmetric centrifugation to incorporate bendamustine into liposomes. According to Table 18 (on page 79), these formulations only provide a marginal increase of stability relative to free bendamustine (20 minutes half-life vs. 14 minutes half-life for free bendamustine).

Accordingly, there is a need for improved formulations of bendamustine which will provide enhanced stability in aqueous solutions.

### SUMMARY OF THE INVENTION

The present invention is directed to pharmaceutical compositions comprising bendamustine and one or more amphiphilic cationic compounds. The compositions self-assemble in an aqueous media to form an aggregate, which aggregates exhibit enhanced stability in aqueous solutions, including plasma. The compositions may further comprise pharmaceutically acceptable excipients, such as sugars, polyalcohols, soluble polymers, salts and lipids. The compositions are suitable for injection or infusion into patients in need for treatment with bendamustine. The unexpectedly enhanced stability afforded by such aggregates permits patients to be treated with bendamustine in lower and/or with less frequent dosages or to improve its therapeutic effect while using the same as presently used treatment protocol.

### DETAILED DESCRIPTION

The present invention is directed to compositions comprising bendamustine and one or more amphiphilic cationic compounds. The composition forms self-assembled aggregates in aqueous media, which aggregates exhibit enhanced stability in aqueous solutions, including plasma.

As is employed herein, the term “amphiphilic” refers to a compound which has at least one hydrophilic moiety and at least one hydrophobic moiety.

The hydrophilic moiety of the amphiphilic compounds useful in the practice of this invention preferably comprises one or more tertiary amine or quarternary ammonium salts. Such cationic groups are typically in the form of a pharmaceutically acceptable salt. It is preferred that the salts are chlorides, sulfates, or sulfonates.

The hydrophobic moiety of the amphiphilic compounds useful in the practice of the present invention comprises a hydrocarbon chain as its hydrophobic part. Such chain may be an aliphatic chain, or a mixed aliphatic-aromatic chain. The aliphatic chain can be linear or branched although it is preferred that the said chain is linear. Preferably, the hydrocarbon chain contains between 8 and 18 carbon atoms.

Preferred tertiary amines include N-alkyl-N,N-dimethylamines, N-alkyl-N,N-diethylamines, N-alkyl-N-N-diethanoloamines, N-alkylmorpholine, N-alkylpiperidine, and N-alkylpyrrolidine. Particularly preferred are sterically hindered tertiary amines, for example, N-alkyl-N-N-diisopropylamine, N-alkylmorpholine, N-alkylpiperidine, and N-alkylpyrrolidine, and other such amines known to those skilled in art.

Preferred quaternary ammonium compounds include N-alkyl-N,N,N-trimethylammonium, N,N-dialkyl-N,N-dimethylammonium, N-alkyl-N-benzyl-NN-diimethylammonium, N-alkyl-pyridinium, N-alkyl-picolinium, alkylamidomethylpyridinium, carbalkoxypyridinium, N-alkylquinolinium, N-alkylisoquinolinium, N,N-alkylmethylpyrollidinium, and 1-alkyl-2,3-dimethylimidazolium.

In one preferred embodiment, the amphiphilic cationic compound is N-cetylpyridinium chloride.

In another preferred embodiment, the amphiphilic cationic compound is N,N-dimethyl-N,N-dioctadecyl ammonium chloride.

In another preferred embodiment, the amphiphilic cationic compound is N,N-dimethyl-N-benzyl-N-octadecyl ammonium chloride.

In another embodiment, the amphiphilic cationic compound is a polycationic polymer having a molecular weight of about 1,000,000 Daltons or less, and comprising multiple cationic groups, where the ratio of the number of cationic groups and the number of atoms in the polymer chain is between 3 and 30.

Polycationic polymers which may be employed include homo-polymers or a co-polymers, including block-co-polymers. Such (co)polymers may be selected from the group consisting of polyolefins, polyethers, polyesters, polyamides, polyurethanes and polysaccharides. Preferred polycationic polymers include quaternized polysaccharides such as Polyquaternium 10 (Hydroxyethylcellulose ethoxylate quaternized), quaternized dextran, and quaternized cyclodextrin.

Other preferred polycationic polymers are shown in Table 1:

TABLE 1

| INCI name | Structure | Product name |
|---|---|---|
| Polyquaternium | Poly[bis(2-chloroethyl]ether-alt- | |
| 2 | 1,3-bis[3-dimethylamino)propyl]- | |
| | urea, quaternized | |
| Polyquaternium | Poly(1-vinylpyrrolidone-co- | Luviquat PQ11 |
| 11 | dimethylammonioethyl | |
| | methacrylate) quaternized | |
| Polyquaternium | Copolymer of vinylpyrrolidone, | Luviquat FC370, |
| 16 and 44 | and quaternized vinylimidazole | Luviquat PC550, |
| | | Luviquat HM 552, |
| | | Luviquat PC 905, |
| | | Luviquat MS 370 |
| Polyquaternium | Copolymer of vinylcaprolactam, | Luviquat Hold |
| 46 | vinylpyrrolidone, and | |
| | quaternized vinylimidazole | |

A mixture of one or more of the amphiphilic cationic compounds described above can be used to prepare the aggregates of the present invention.

The aggregates of the present invention are typically prepared by mixing the amphiphilic cationic compound with bendamustine. The cationic agent is a chemical, the molecules of which self-assemble to form aggregates in aqueous media. Such aggregates have lipophilic core and ionic outer layer.

The weight ratios of bendamustine to amphiphilic cationic compound may range from about 1:1 to about 1:1000; is preferably between about 1:1 and about 1:100; and is most preferably between about 1:3 and about 1:30.

As will be recognized by those of skill in the art, the concentration of amphiphilic cationic compounds must be at or above the critical micelle concentration during application. The critical micelle concentration will depend upon a number of factors, including the composition of the amphiphilic cationic compounds, pH, ion strength, and the like, but can easily be determined by routine experimentation for any given composition employing procedures well known to one of skill in the art.

The compositions of the present invention may further contain pharmaceutically acceptable excipients, such as sugars, polyalcohols, soluble polymers, salts and lipids.

Sugars and polyalcohols which may be employed include, without limitation, lactose, sucrose, mannitol, and sorbitol.

Illustrative of the soluble polymers which may be employed are polyoxyethylene, poloxamers, polyvinylpyrrolidone, and dextran.

Useful salts include, without limitation, sodium chloride, magnesium chloride, and calcium chloride.

Lipids which may be employed include, without limitation, fatty acids, glycerol fatty acid esters, glycolipids, and phospholipids.

Compositions of bendamustine and amphiphilic cationic compounds may disperse and release the drug upon dilution in aqueous media.

The composition of bendamustine with amphiphilic cationic compounds renders the bendamustine sufficiently chemically stable.

The compositions of the present invention are suitable for injection or infusion into patients in need for treatment with bendamustine.

The invention can be further illustrated by the following examples thereof, although it will be understood that these examples are included merely for purposes of illustration and are not intended to limit the scope of the invention unless otherwise specifically indicated. All percentages, ratios, and parts herein, in the Specification, Examples, and Claims, are by weight and are approximations unless otherwise stated.

### EXAMPLES

### Example 1

### Preparation of Bendamustine Composition Comprising Cetylpyridinium Chloride

0.5 g Cetylpyridinium chloride was dissolved in 100 mL water. This solution was used to dissolve solid mixture of 60 mg of bendamustine and 102 mg of D-mannitol. The composition was thoroughly mixed.

### Example 2

### Preparation of Bendamustine Composition Comprising Polyquaternium 46

5 g of 20% Polyquaternium 46 was diluted with 95 mL water. This solution was used to dissolve solid mixture of 60 mg of bendamustine and 102 mg of D-mannitol. The composition was thoroughly mixed.

### Example 3

### Bendamustin Chemical Stability in Various Formulations Comprising Amphilphilic Cationic Agents and Phosphate Buffer

The formulations listed in Table 2 below were prepared by dissolving bendamustine hydrochloride in a pre-equilibrated aqueous solution of amphiphilic cationic compound containing 6 mM phosphate buffer, pH 7.2. The final concentration of bendamustine was 0.6 mg/mL. The formulation was incubated at 25° C., and was periodically analyzed by HPLC as follows. 10 μL samples were separated using Waters SymmetryShield RP-18 3.5 μm column (4.6×50 mm) at the flow of 1.5 mL/min of acetonitrile-water gradient containing 0.1% TFA. Peak detection has been performed with means of UV absorption detection at 260 nm. The area of the peak of bendamustine was used to evaluate the rate of drug decomposition in the first order kinetics model. The results expressed as decomposition half times (T½) are presented in Table 2 below.

TABLE 2

| | |
|---|---|
| Amphiphilic cationic agent | T½ |

| | | |
|---|---|---|
| Control (phosphate buffer) | 44 | min |
| Cetylpyridinium chloride 0.5% | 3031 | min |
| Benzyldimethyldodecylammonium chloride 0.5% | 1576 | min |
| Dodecylpyridinium chloride 0.4% | 997 | min |
| Hexadecyltrimethylammonium chloride 0.5% | 2278 | min |
| Benzyldimethyltetradecylammonium chloride 0.5% | 2329 | min |
| Octedecyldimethylbenzylammonium chloride 0.25% | 2081 | min |
| Domiphen bromide 0.5% | 2843 | min |

The above examples demonstrate the unexpected stability exhibited by the aggregates of this invention.

It is to be understood that the above-described embodiments are illustrative of only a few of the many possible specific embodiments, which can represent applications of the principles of the invention. Numerous and varied other arrangements can be readily devised in accordance with these principles by those skilled in the art without departing from the spirit and scope of the invention.

What is claimed is:

1. A composition comprising bendamustine and an amphiphilic cationic compound.

2. The composition of claim 1 wherein a hydrophilic part of the amphiphilic cationic compound comprises a tertiary amine or quarternary ammonium salt.

3. The composition of claim 1 wherein the weight ratio of bendamustine to amphiphilic cationic compound is between about 1:1 and about 1:1000.

4. The composition of claim 1 wherein the weight ratio of bendamustine to amphiphilic cationic compound is between about 1:1 and about 1:100.

5. The composition of claim 1 wherein the weight ratio of bendamustine to amphiphilic cationic compound is between about 1:3 and about 1:30.

6. The composition of claim 1 wherein a hydrophilic part of the amphiphilic cationic compound comprises a tertiary amine.

7. The composition of claim 1 wherein the hydrophilic part of the amphiphilic cationic compound comprises a sterically hindered tertiary amine.

8. The composition of claim 1 wherein the amphiphilic cationic compound is selected from the group consisting of N-alkyl-N-N-diisopropylamine, N-alkylmorpholine, N-alkylpiperidine, and N-alkylpyrrolidine.

9. The composition of claim 1 wherein a hydrophilic part of the amphiphilic cationic compound comprises a quarternary ammonium salt.

10. The composition of claim 1 wherein the amphiphilic cationic compound comprises a N-alkyl-N,N,N-trimethylammonium, N,N-dialkyl-N,N-dimethylammonium, N-alkyl-N-benzyl-NN-diimethylammonium, N-alkyl-pyridinium, N-alkyl-picolinium, alkylamidomethylpyridinium, carbalkoxypyridinium, N-alkylquinolinium, N-alkylisoquinolinium, N,N-alkylmethylpyrollidinium, or a 1-alkyl-2,3-dimethylimidazolium salt.

11. The composition of claim 1 wherein the amphiphilic cationic compound is N-cetylpyridinium chloride.

12. The composition of claim 1 wherein the amphiphilic cationic compound is N,N-dimethyl-N,N-dioctadecyl ammonium chloride.

13. The composition of claim 1 wherein the amphiphilic cationic compound is N,N-dimethyl-N-benzyl-N-octadecyl ammonium chloride.

14. The composition of claim 1 wherein the amphiphilic cationic compound is a polycationic polymer having a molecular weight of about 1,000,000 Daltons or less.

15. The composition of claim 14 wherein the polycationic polymer is selected from the group consisting of polyolephins, polyethers, polyesters, polyamides, polyurethanes and polysaccharides.

16. The composition of claim 1 wherein the amphiphilic cationic compound is selected from the group consisting of Polyquaternium 10, quaternized dextran, quaternized cyclodextrin, Polyquaternium 2, Polyquaternium 11, Polyquaternium 16, Polyquaternium 44, and Polyquaternium 46.

17. The composition of claim 1 wherein the composition is a self assembled aggregate.

18. The composition of claim 1 wherein the composition is a pharmaceutical composition.
